(19) United States
(12) Patent Application Publication
(10) Pub. No.: US 2024/0307260 A1
(43) Pub. Date: Sep. 19, 2024

(54) TREATMENT TIP AND SYSTEM FOR TREATING VAGINAL CAVITY

(71) Applicant: SHENZHEN PENINSULA MEDICAL GROUP, Shenzhen (CN)

(72) Inventors: Yi DING, Shenzhen (CN); Jian LI, Shenzhen (CN); Shangkuai HUANG, Shenzhen (CN); Yanan LI, Shenzhen (CN); Xiaobing LEI, Shenzhen (CN)

(73) Assignee: SHENZHEN PENINSULA MEDICAL GROUP, Shenzhen (CN)

(21) Appl. No.: 18/648,747

(22) Filed: Apr. 29, 2024

Related U.S. Application Data
(63) Continuation of application No. PCT/CN2022/103539, filed on Jul. 4, 2022.

(30) Foreign Application Priority Data
Oct. 29, 2021 (CN) 202111280431.4

(51) Int. Cl. A61H 19/00; A61H 9/00; A61N 1/05

(52) U.S. Cl. CPC A61H 19/44; A61H 9/0078; A61H 2201/0153; A61H 2201/10; A61H 2201/1409; A61H 2201/1685; A61N 1/0524

(57) ABSTRACT

Disclosed are a treatment tip and a system for treating vaginal cavity. The treatment tip is configured to be detachably connected to a handle and the treatment tip includes a flexible shell and a support component. The flexible shell is inflatable. The support component is installed in the flexible shell to support the flexible shell.

### CROSS-REFERENCE TO RELATED APPLICATIONS

The present application is a continuation application of International Application No. PCT/CN2022/103539, filed on Jul. 4, 2022, which claims priority to Chinese Patent Application No. 202111280431.4, filed on Oct. 29, 2021. The disclosures of the above-mentioned applications are incorporated herein by reference in their entireties.

### TECHNICAL FIELD

The present application relates to the technical field of medical instruments, and in particular, to a treatment tip and a system for treating vaginal cavity.

### BACKGROUND

In the related art, the support component of the cavity treatment system is integrated with the handle, and the flexible shell is set on the support component and bonded to the handle. Since the treatment tip needs to be inserted into the cavity, it is a personal item. After completing the course of treatment, the cavity treatment system is no longer needed, but it may not be used by others, causing the cavity treatment system to be idle, resulting in waste.

### SUMMARY

The main purpose of the present application is to provide a treatment tip, which aims to reduce waste.

In order to achieve the above purpose, the present application provides a treatment tip. The treatment tip is configured to be detachably connected to a handle, and the treatment tip includes a flexible shell being inflatable; and a support component installed in the flexible shell to support the flexible shell.

In an embodiment, the treatment tip further includes a hard circuit board and a plurality of electrode sheets; the hard circuit board is disposed on the flexible shell to support the flexible shell, and the electrode sheets are disposed on the hard circuit board and exposed outside an outer peripheral wall of the flexible shell; the plurality of electrode sheets are provided at intervals along a length direction of the flexible shell.

In an embodiment, the flexible shell is provided with an inflatable cavity, and the support component includes a support portion extending into the inflatable cavity to support the flexible shell.

In an embodiment, the treatment tip further includes an installation seat installed on the flexible shell, and the installation seat is provided with an inflation hole communicated with the inflatable cavity.

In an embodiment, the treatment tip further includes a sealing gasket installed on the installation seat, and the sealing gasket includes a gasket body and a sealing ring; the gasket body is provided on a side of the installation seat away from the handle, and the sealing ring is configured to extend along a wall surface of the inflation hole and is disposed around the inflation hole.

In an embodiment, the sealing ring is provided with an outer flange.

In an embodiment, the treatment tip further includes a sealing member, and the sealing member is elastically abutted against the sealing gasket to seal the inflation hole.

In an embodiment, the flexible shell is provided with a port communicated with the inflatable cavity, and the port is provided with an extension ring; the installation seat is provided with a receiving groove for receiving the extension ring, and a periphery of the receiving groove is provided with an annular protrusion for abutting against the flexible shell.

In an embodiment, a plurality of annular protrusions are provided at intervals around a periphery of the installation seat.

In an embodiment, the treatment tip further includes an installation member installed at the port; the installation member includes an extension barrel for connecting the installation seat, and a thimble embedded in the flexible shell.

In an embodiment, the support component further includes a base installed on the installation seat; the support portion is provided on the base, and the installation member is configured to pass through the base and is engaged with the installation seat.

In an embodiment, one of the installation member and the installation seat is provided with a hook piece, and the other of the installation member and the installation seat is provided with a buckle piece for cooperating with the hook piece.

In an embodiment, the treatment tip further includes an adapter plate, and the adapter plate is installed on the support component; the adapter plate is electrically connected to the hard circuit board, and the adapter plate is configured to electrically connect with the handle.

The present application further provides a system for treating vaginal cavity, including an above-mentioned treatment tip and a handle. The treatment tip includes a flexible shell and a support component. The flexible shell is inflatable, and the support component installed in the flexible shell to support the flexible shell.

In an embodiment, the handle is provided with a limiting pillar configured to extend into and out of the treatment tip; the treatment tip is provided with a limiting hole for accommodating the limiting pillar, and the limiting hole is offset from a rotation axis of the handle.

In an embodiment, one of the treatment tip and the handle is provided with a snap block, and the other of the treatment tip and the handle is provided with a snap slot.

In an embodiment, the handle is provided with an installation groove, and the installation groove is configured to accommodate at least part of the treatment tip; a groove wall of the installation groove is provided with the snap slot, and the treatment tip is provided with the snap block; and
the snap slot includes a channel slot for the snap block to extend into and a clamping slot extending along a circumference of the handle, and the channel slot is communicated with the clamping slot.

In an embodiment, the flexible shell is provided with an inflatable cavity, the treatment tip further includes an installation seat installed on the flexible shell, and the installation seat is provided with an inflation hole communicated with the inflatable cavity; the handle is provided with an abutting pillar extending into the inflation hole, and the abutting pillar is provided with a gas delivery port.

In an embodiment, the treatment tip further includes a sealing gasket installed on the installation seat, and the sealing gasket includes a gasket body and a sealing ring; the gasket body is provided on a side of the installation seat away from the handle, and the sealing ring is configured to extend along a wall surface of the inflation hole and is exposed at the inflation hole.

In an embodiment, the treatment tip further includes a sealing member, and the sealing member is elastically abutted against the sealing gasket to seal the inflation hole; in response to that the abutting pillar extends into the inflation hole, the abutting pillar is abutted against the sealing member to open the inflation hole.

The flexible shell of the technical solution of the present application may be inflated, and the treatment tip and the handle are detachably connected. Therefore, after the treatment tip is damaged or the service life of the treatment tip expires, only a new treatment tip may be replaced, making full use of the handle, avoiding material waste, and improving the service life of the product. At the same time, when treatment projects are carried out in a hospital, the hospital provides handles, and each user may purchase the treatment tip for his or her own use. That is, a plurality of treatment tips may use the same handle. There is no need for each treatment tip to be equipped with a handle, which saves user treatment costs, and prevents the handle from being idle. In addition, different types of treatment tips may be replaced, and the product has a wide range of applications to ensure that the electrode pads of the treatment tip fit well with the treatment area, thereby improving the treatment effect and reducing the risk of scalding the treatment area.

### BRIEF DESCRIPTION OF THE DRAWINGS

In order to more clearly illustrate the technical solutions in the embodiments of the present application or in the related art, a brief introduction will be given to the accompanying drawings required in the description of the embodiments or the related art. Obviously, the accompanying drawings in the following description are only an embodiment of the present application. For those skilled in the art, other accompanying drawings may be obtained based on the structures shown in these drawings without any creative effort.

FIG. 1 is a schematic structural view of a system for treating vaginal cavity according to an embodiment of the present application.

FIG. 2 is a schematic structural view of a treatment tip according to an embodiment of the present application.

FIG. 3 is a schematic structural view of a flexible shell according to an embodiment of the present application.

FIG. 4 is a schematic cross-sectional view of the flexible shell and an installation component according to an embodiment of the present application.

FIG. 5 is an enlarged view of a portion A in FIG. 4.

FIG. 6 is an enlarged view of a portion B in FIG. 4.

FIG. 7 is a schematic view of electrode sheets, a hard circuit and a first flexible circuit board according to an embodiment of the present application.

FIG. 8 is a schematic view of the electrode sheets according to an embodiment of the present application.

FIG. 9 is a schematic view of the electrode sheets from another perspective according to an embodiment of the present application.

FIG. 10 is a schematic view of the installation component according to an embodiment of the present application.

FIG. 11 is a schematic view of the installation component in FIG. 10 from another perspective.

FIG. 12 is a schematic view of an installation seat and a support component according to an embodiment of the present application.

FIG. 13 is a exploded view of the installation seat and the support component in FIG. 12.

FIG. 14 is a schematic view of the installation seat and the support component in FIG. 13 from another perspective.

FIG. 15 is a schematic view of the installation seat according to an embodiment of the present application.

FIG. 16 is a schematic view of the installation seat in FIG. 15 from another perspective.

FIG. 17 is a schematic view of a sealing gasket according to an embodiment of the present application.

FIG. 18 is a schematic view of the sealing gasket in FIG. 17 from another perspective.

FIG. 19 is a schematic view of a second flexible circuit board according to an embodiment of the present application.

FIG. 20 is a schematic view of a sealing member according to an embodiment of the present application.

FIG. 21 is a schematic cross-sectional view of the system for treating vaginal cavity (assembled) according to an embodiment of the present application.

FIG. 22 is an enlarged view of a portion C in FIG. 21.

FIG. 23 is an enlarged view of a portion D in FIG. 22.

FIG. 24 is a schematic view of the hard circuit and the first flexible circuit board according to an embodiment of the present application.

FIG. 25 is a schematic view of the hard circuit and the first flexible circuit board in FIG. 24 from another perspective.

FIG. 26 is a schematic view of an installation member according to another embodiment of the present application.

FIG. 27 is a schematic view of the system for treating vaginal cavity according to another embodiment of the present application.

FIG. 28 is an enlarged view of a portion E in FIG. 27.

FIG. 29 is a schematic view of the installation seat and the support component according to another embodiment of the present application.

FIG. 30 is a schematic view of the support component in FIG. 29.

FIG. 31 is a schematic structural view of the treatment tip according to another embodiment of the present application.

FIG. 32 is a schematic structural view of a handle according to an embodiment of the present application.

FIG. 33 is a schematic view of the handle in FIG. 32 from another perspective.

FIG. 34 is a schematic cross-sectional view of the handle in FIG. 32.

FIG. 35 is an enlarged view of a portion F in FIG. 34.

FIG. 36 is a schematic cross-sectional view of the system for treating vaginal cavity (disassembled) according to an embodiment of the present application.

FIG. 37 is an enlarged view of a portion G in FIG. 36.

The realization of the purpose, functional characteristics and advantages of the present application will be further described with reference to the attached drawings in combination with embodiments.

### DETAILED DESCRIPTION OF THE EMBODIMENTS

The technical solutions of embodiments of the present application will be clearly and completely described with reference to the drawings in the embodiments of the present application. Obviously, the described embodiments are only some rather than all of the embodiments of the present application. Based on the embodiments of the present application, all other embodiments obtained by those skilled in the art without creative efforts shall fall within the scope of the present application.

It should be noted that if there are directional indications (such as up, down, left, right, front, rear, etc.) in the present application, the directional indications are only used to explain the relative positional relationship, movement situation, etc. among components in a specific attitude (as shown in the drawings). If the specific attitude changes, the directional indication also changes accordingly.

In addition, the descriptions related to “first”, “second” and the like in the present application are merely for descriptive purposes, and should not be understood as indicating or implying their relative importance or implicitly indicating the number of technical features indicated. Therefore, the features defined by “first” and “second” may explicitly or implicitly include at least one such feature. In addition, “and/or” in the whole text includes three solutions, taking A and/or B as an example, including A technical solution, or B technical solution, or a technical solution that both A and B meet. Besides, the technical solutions among various embodiments may be combined with each other, but the combination must be based on what may be achieved by those skilled in the art. When the combination of technical solutions is contradictory or may not be achieved, it should be considered that such combination does not exist, and is not within the scope of the present application.

The present application is to provide a system for treating vaginal cavity.

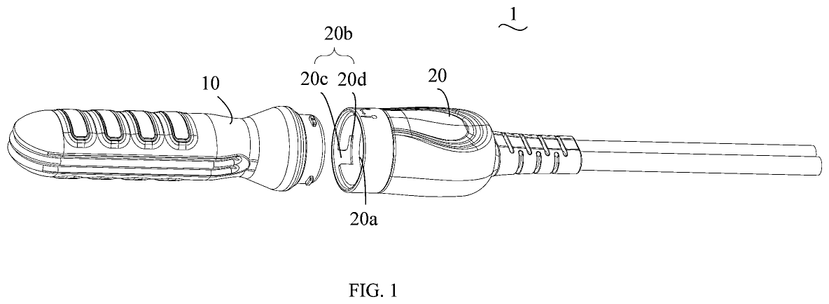

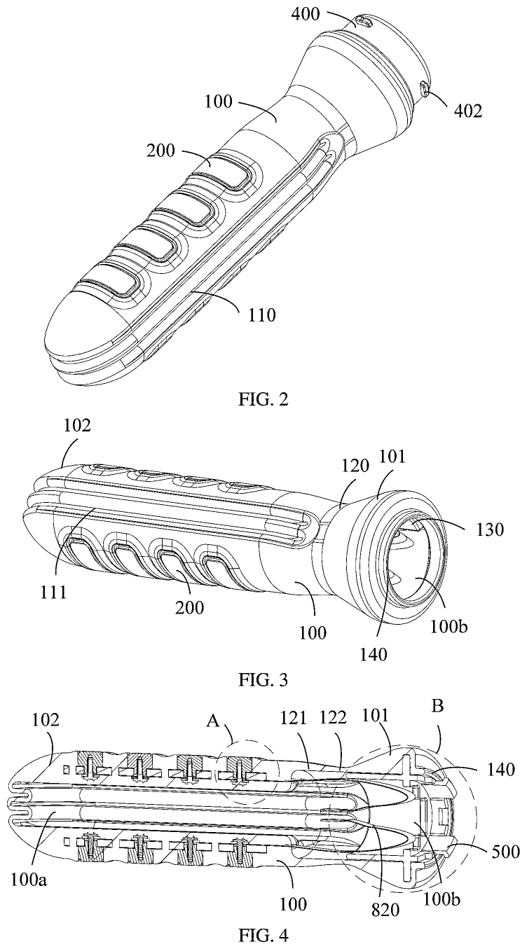

In an embodiment of the present application, as shown in FIG. 1, FIG. 2 and FIG. 12, the system for treating vaginal cavity 1 includes a handle 20 and a treatment tip 10. The treatment tip 10 includes a flexible shell 100 and a support component 600 installed on the flexible shell 100. The flexible shell 100 may be inflated and expanded. The treatment tip 10 is detachably connected to the handle 20.

In the technical solution of the present application, the flexible shell 100 of may be inflated and expanded, and the treatment tip 10 is detachably connected to the handle 20. Therefore, after the treatment tip 10 is damaged, only the treatment tip 10 may be replaced with a new one, and the handle 20 may be fully utilized, thereby avoiding the waste of materials and improving the service life of the product. At the same time, each user may use a treatment tip 10 independently, that is to say, plurality of treatment tips 10 may be use with the same handle 20, and there is no need for each treatment tip 10 to be equipped with a handle 20, which saves the user's treatment costs and avoids wasting handles 20.

Specifically, as shown in FIG. 2 to FIG. 3, the treatment tip 10 includes a flexible shell 100. The flexible shell 100 is used to extend into the cavity for treatment. The flexible shell 100 may be in regular or irregular shapes such as spherical, cylindrical, or tubular, or may be a combination of plurality of regular shapes. As shown in FIG. 3 to FIG. 4, The flexible shell 100 extends along a straight line and has a first end 101 for connecting the handle 20 and a second end 102 opposite to the first end 101. The flexible shell 100 is a flexible part, which is friendly to the inner wall of the cavity and has a weak sense of foreign matter, making it easy for users to accept. The flexible shell 100 is made of various materials, such as silicone, rubber, etc.

As shown in FIG. 3, the treatment tip 10 also includes an electrode sheet 200. The electrode sheet 200 is exposed on the outer peripheral wall of the flexible shell 100, thereby contacting the inner wall of the user's vaginal cavity to perform treatment, such as heating or electrical stimulation. The electrode sheet 200 may protrude from the surface of the flexible shell 100 so that the electrode sheet 200 may easily contact the inner wall of the vaginal cavity. As shown in FIG. 2 to FIG. 3, the flexible shell 100 may be inflated to increase the volume of the flexible shell 100, so that the electrode sheet 200 on the flexible shell 100 may be more easily attached to the inner wall of the vaginal cavity to achieve treatment, which avoids the electrode sheet 200 being unable to adhere to the vaginal cavity and affecting treatment caused by the inner diameter of the cavity of some users being too large.

There are many ways to realize the expansion of the flexible shell 100. After the flexible shell 100 is inflated, the flexible shell 100 is expanded and the thickness of the flexible shell 100 is thinned. As shown in FIG. 2 to FIG. 3, in one embodiment, the outer wall surface of the flexible shell 100 is provided with a corrugated portion 110, so that the flexible shell 100 has an uninflated compressed state and an inflated expanded state. As shown in FIG. 2 and FIG. 3, in the compressed state, the flexible shell 100 is not inflated, and the corrugated portion 110 is in a shrunken state, the overall volume of the flexible shell 100 is small, making it easy to extend the flexible shell 100 into the vaginal cavity and reducing the user's foreign body sensation. When the flexible shell 100 is inflated, the corrugated portion 110 is opened, and the flexible shell 100 is in an expanded state, so that the electrode sheet 200 on the flexible shell 100 may be easily attached to the inner wall of the vaginal cavity.

As shown in FIG. 3, in an embodiment, the corrugated portion 110 may include a plurality of grooves 111 formed on the outer wall surface of the flexible shell 100. The plurality of grooves 111 extend along the length direction of the flexible shell 100, so that the outer diameter of the flexible shell 100 may increase and the volume may increase evenly when the flexible shell 100 is in an inflated and expanded state. In order to fully increase the volume of the flexible shell 100, as shown in FIG. 2 to FIG. 3, in an embodiment, the flexible shell 100 is provided with two corrugated portions 110, and the two corrugated portions 110 are respectively located on two opposite sides flexible shell 100 and intersect at the second end 102 of the flexible shell 100, so that the volume of the entire flexible shell 100 may be increased evenly, which is beneficial to the contact between the electrode sheet 200 and the inner wall of the vaginal cavity.

As shown in FIG. 3, a plurality of electrode sheets 200 are provided. The plurality of electrode sheets 200 are provided at intervals along the length direction of the flexible shell 100, thereby increasing the treatment sites of the treatment tip 10. The electrode sheets 200 on the flexible shell 100 may be formed into plurality of groups. Each group of electrode sheets 200 includes a plurality of electrode sheets 200. The plurality of groups of electrode sheets 200 may be provided at intervals along the circumferential direction of the flexible shell 100. As shown in FIG. 2 to FIG. 3. Two groups of electrode sheets 200 are formed on the flexible shell 100. The two groups of electrode sheets 200 are located on opposite sides of the flexible shell 100. The corrugated portion 110 is located between the two groups of electrode sheets 200.

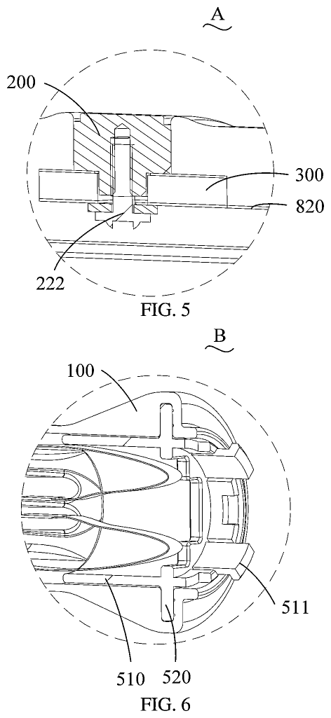

As shown in FIG. 4 to FIG. 5 and FIG. 7, the treatment tip 10 also includes a hard circuit board 300. The hard circuit board 300 is installed on the flexible shell 100 to support the flexible shell 100, so that the flexible shell 100 may maintain its own shape, and the rigidity of the flexible shell 100 is improved. In this way, the flexible shell 100 may be pushed into the vaginal cavity under force. The hard circuit board 300 may be installed in the flexible shell 100 and is not exposed on the wall surface of the inflatable cavity 100a of the flexible shell 100. The hard circuit board 300 may also be installed in the flexible shell 100 and flush with the cavity wall of the inflatable cavity 100a of the flexible shell 100, or the hard circuit board 300 is installed on the inner wall of the flexible shell 100.

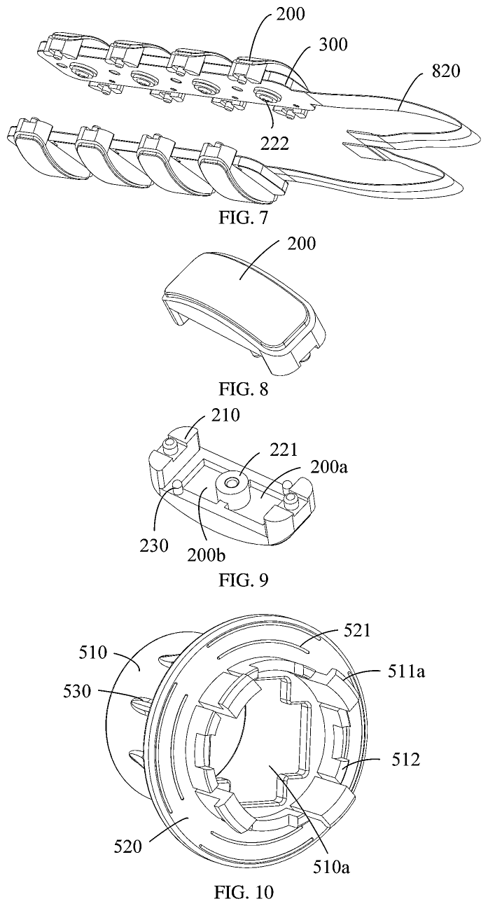

As shown in FIG. 7, the electrode sheet 200 is installed on the hard circuit board 300. The hard circuit board 300 supports the electrode sheet 200. When the flexible shell 100 is in a compressed state, the electrode sheet 200 is located outside the hard circuit board 300 to prevent the electrode sheet 200 from sinking into the flexible shell 100; when the flexible shell 100 is in an expanded state, the gas in the inflatable cavity 100a of the flexible shell 100 will have an outward thrust to the hard circuit board 300. Under the action of the thrust, the hard circuit board 300 contacts the electrode sheet 200 outwards, making it easier for the electrode sheet 200 to contact the inner wall of the vaginal cavity.

As shown in FIG. 4 and FIG. 7, in an embodiment, the hard circuit board 300 extends along the length direction of the flexible shell 100 to support the flexible shell 100 and the electrode sheet 200. The plurality of electrode sheets 200 may be installed on different hard circuit boards 300 or on the same circuit board. As shown in FIG. 7, in an embodiment, the plurality of electrode sheets 200 may be installed on the same hard circuit board 300. The electrode sheet 200 on the same hard circuit board 300 may be evenly stressed, which facilitates the contact between the electrode sheet 200 and the inner wall of the vaginal cavity to ensure the therapeutic effect of the treatment tip 10, and may also reduce the number of assembly parts to facilitate installation by workers, thereby improving the assembly efficiency of the treatment tip 10.

As shown in FIG. 7, when the flexible shell 100 is provided with a plurality of groups of electrode sheets 200, each group of electrode sheets 200 may be installed on the same hard circuit board 300, so that each group of electrode sheets 200 is subject to an roughly equivalent supporting force and an roughly equivalent contact force from the hard circuit board 300, which facilitates the contact between the electrode sheet 200 and the inner wall of the cavity.

As shown in FIG. 7, in an embodiment, when the flexible shell 100 is provided with two groups of electrode sheets 200, two hard circuit boards 300 may be provided, and each hard circuit board 300 corresponds to one group of electrode sheets 200. The two hard circuit boards 300 may be provided opposite to each other on the flexible shell 100, which not only enables the treatment tip 10 to treat the opposite sides of the inner wall of the vaginal cavity, but also makes the electrode sheet 200 on the flexible shell 100 bear force evenly, thereby facilitating contact between the electrode sheet 200 and the inner wall of the vaginal cavity.

There are many ways to install the electrode sheet 200 on the hard circuit board 300. As shown in FIG. 7, FIG. 8 and FIG. 9, in an embodiment, the electrode sheet 200 is provided with a limiting groove 200a. The hard circuit board 300 is installed in the limiting groove 200a.

Specifically, as shown in FIG. 7 and FIG. 9, the electrode sheet 200 has two press fit portions 210 protruding from a side facing the inflatable cavity 100a of the flexible shell 100. The two press fit portions 210 are spaced apart, and a limiting groove 200a for accommodating the hard circuit board 300 is formed between the two press fit portions 210, so that the electrode sheet 200 is installed on the hard circuit board 300 through the limiting groove 200a. The hard circuit board 300 is installed on the flexible shell 100, and the press fit portion 210 limits the electrode sheet 200 to prevent the electrode sheet 200 from rotating and plays a positioning role.

As shown in FIG. 7 and FIG. 9, in an embodiment, the hard circuit board 300 may be installed at the bottom of the limiting groove 200a. The limiting groove 200a is provided with a connection portion 221 at the bottom of the groove. The treatment tip 10 may also include a connector 222, which passes through the hard circuit board 300 and is connected to the connection portion 221 for installing hard circuit board 300 and the electrode sheet 200. There are many ways to connect the connector 222 to the connection portion 221. As shown in FIG. 9, the connection portion 221 is protruding from the bottom of the limiting groove 200a. The connection portion 221 is provided with a threaded hole. The connector 222 is provided with external threads. The connection portion 221 is screwed to the connector 222, so that the electrode sheet 200 is installed on the hard circuit board 300. It may be understood that the connection portion 221 may be integrally formed on the electrode sheet 200, thereby improving the stability of the connection portion 221. The connection portion 222 may be a screw nail.

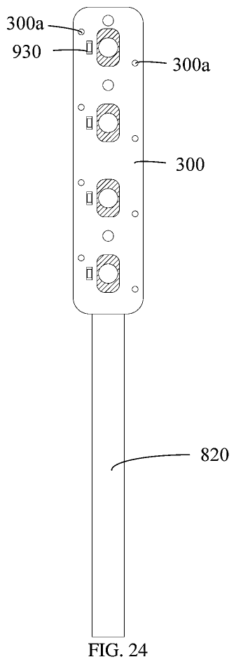

In order to prevent the hard circuit board 300 from being installed upside down, as shown in FIG. 9, in one embodiment, two first positioning pillars 230 are provided at the bottom of the limiting groove 200a. The positioning pillar 230 is disposed diagonally at the bottom of the limiting groove 200a. As shown in FIG. 24, the hard circuit board 300 is provided with a positioning hole 300a that is matched with the first positioning pillar 230. The first positioning pillar 230 is inserted into the positioning hole 300a, so that the hard circuit board 300 may cooperate with the electrode sheet 200 according to a certain installation orientation, which plays a role in fool-proof design of the hard circuit board 300, preventing workers from rework, and improving the assembly efficiency of the product.

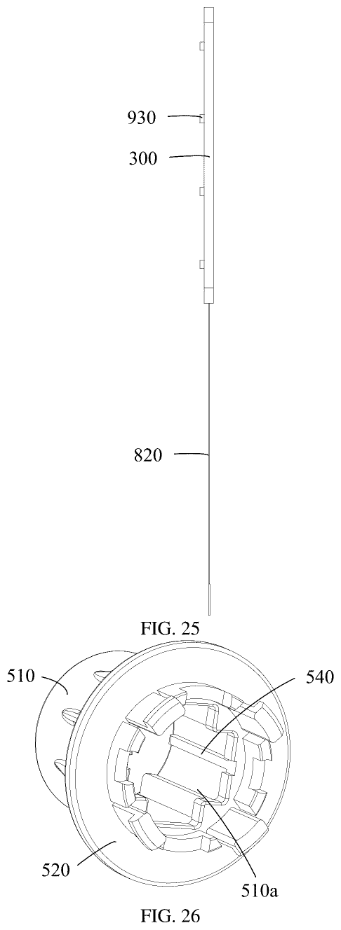

As shown in FIG. 24, FIG. 25 and FIG. 9, in an embodiment, a temperature sensor 930 is installed on the side of the hard circuit board 300 facing the bottom of the limiting groove 200a of the electrode sheet 200. The bottom of the limiting groove 200a is also provided with an avoidance groove 200b for accommodating the temperature sensor 930. In order to prevent the temperature sensor 930 from being in rigid contact with the electrode sheet 200 and damaging the temperature sensor 930, a thermally conductive silica gel may be installed in the avoidance groove 200b, and the temperature sensor 930 is in elastic contact with the silica gel, thereby protecting the temperature sensor 930, At the same time, the silica gel may transfer the heat of the electrode sheet 200 to the temperature sensor 930, allowing the temperature sensor 930 to recognize the induction, which is beneficial to adjusting the temperature of the electrode sheet 200 in a timely manner and preventing the temperature of the electrode sheet 200 from being too high or too low.

The electrode sheet 200 is electrically connected to the hard circuit board 300. As shown in FIG. 4 and FIG. 7, in an embodiment, the treatment tip 10 also includes a first flexible circuit board 820. The first flexible circuit board 820 is provided with a plurality of signal lines, and each signal line is connected to the hard circuit board 300. As shown in FIG. 7, the first flexible circuit board 820 is installed on the hard circuit board 300, and a plurality of signal lines are provided on the first flexible circuit board 820 to prevent the plurality of signal lines from being entangled with each other, thereby, improving the cleanliness of the product. The first flexible circuit board 820 may be installed on the side of the hard circuit board 300 facing away from the electrode sheet 200, or may be installed on the side of the hard circuit board 300 facing the electrode sheet 200. In addition, the first flexible circuit board 820 may be connected with other devices, thereby improving the assembly efficiency of the treatment tip 10.

In order to prevent the flexible shell 100 from leaking water and air, as shown in FIG. 4 and FIG. 7, to ensure the sealing of the flexible shell 100, the flexible shell 100 is made by liquid silicone injection molding, and the hard circuit board 300 is attached to the flexible shell 100 and becomes a part of the shell wall of the flexible shell 100. Specifically, during the molding process of the flexible shell 100, the electrode sheet 200 and the hard circuit board 300 are integrally positioned in the molding die, and the electrode sheet 200 is compressed by the compression mold. The liquid silicone is injected into the molding mold through an extrusion force, thereby obtaining an integrated structure of the electrode sheet 200, the hard circuit board 300 and the flexible shell 100. It may be understood that by molding the electrode sheet 200, the hard circuit board 300 and the flexible shell 100 into an integrated structure through liquid silicone injection, not only the sealing of the flexible shell 100 may be ensured, but also the stability of the connection between the electrode sheet 200 and the flexible shell 100 may be ensured, which prevents the flexible shell 100 from frequently expanding and contracting, causing the electrode 200 to fall off from the flexible shell 100. Before the hard circuit board 300 is positioned in the mold, the plurality of electrode sheets 200, the hard circuit board 300 and the first flexible circuit board 820 may be assembled first, and then the liquid silicone injection molding is performed to realize the assembly and processing of the flexible shell 100.

As shown in FIG. 2 and FIG. 4, in an embodiment, the treatment tip 10 also includes an installation member 500 and an installation seat 400. The flexible shell 100 is installed on the installation seat 400 through the installation member 500. The installation seat 400 is used for connecting the handle 20 so that the flexible shell 100 is installed on the handle 20.

As shown in FIG. 4, the first end 101 of the flexible shell 100 is provided with a port 100b connected to the inflatable end. The installation member 500 is installed at the port 100b of the flexible shell 100. Specifically, the installation member 500 includes an extension barrel 510. The extension barrel 510 at least partially extends into the inflatable cavity 100a. At the same time, the other end of the extension barrel 510 at least partially protrudes from the port 100b, and the part of the extension barrel 510 protruding from the port 100b may be connected with the installation seat 400.

As shown in FIG. 4 and FIG. 6, the extension barrel 510 is provided in a cylindrical shape, and may be cylindrical or square cylindrical. Specifically, the extension barrel 510 may be set according to the shape of the port 100b. The extension barrel 510 may extend in the direction towards the second end 102 of the flexible shell 100. One end of the extension barrel 510 extends into the inflatable cavity 100a, and the other end protrudes at the port 100b of the flexible shell 100, so as to facilitate the connection between the extension barrel 510 and the installation seat 400. In this way, the flexible shell 100 is installed on the installation seat 400. As shown in FIG. 6, the extension barrel 510 extends along the inner wall of the flexible shell 100, and the flexible shell 100 clamps the extension barrel 510 to play a positioning role.

As shown in FIG. 4 and FIG. 6, the installation member 500 also includes a thimble 520. The thimble 520 protrudes from the side of the extension barrel 510. The inner wall of the inflatable cavity 100a is provided with a thimble slot that matches the thimble 520. By embedding thimble 520 in the thimble slot, the installation member 500 may be easily installed on the port 100b of the flexible shell 100. When the second end 102 of the flexible shell 100 expands and deforms, because the thimble 520 is embedded in the flexible shell 100, the installation member 500 is not easy to fall off from the port 100b. The connection between the flexible shell 100 and the installation member 500 is stable, highly reliable and practical.

In order to further improve the stability of the connection of the flexible shell 100, as shown in FIG. 4, in an embodiment, the corrugated portion 110 on the flexible shell 100 extends from the second end 102 to the front side of the first end 101, also that is, the first end 101 is not provided with the corrugated portion 110. Thus, when the flexible shell 100 is in an expanded state, the first end 101 expands to a smaller extent, making the thimble 520 less likely to fall out of the thimble slot. The stability of the connection between the installation member 500 and the flexible shell 100 is further improved.

It may be understood that the thimble 520 and the extension barrel 510 may be provided integrally or separately. As shown in FIG. 10, in an embodiment, the thimble 520 is integrally formed with the extension barrel 510, thereby improving the stability of the installation member 500 itself. The width of the thimble 520 and the extension barrel 510 may be the same or different, and may be set according to the actual situation of the flexible shell 100.

As shown in FIG. 4 and FIG. 10, in an embodiment, the thimble 520 extends along the thickness direction of the flexible shell 100, so that the thimble 520 is directly and deeply embedded in the flexible shell 100. It is more difficult to pull the installation member 500 out from the port 100b, which improves the stability of the connection between the installation member 500 and the flexible shell 100. The installation member 500 may be a hardware piece, and the installation seat 400 is also a hardware piece, so that the two hardware pieces are rigidly connected and the reliability of the connection is improved.

During the treatment process, the treatment tip 10 needs to be supported by hands, otherwise the treatment tip 10 will easily prolapse from the cavity, affecting the treatment effect. In order to solve this problem, as shown in FIG. 3 to FIG. 4, in an embodiment, a fall-off prevention portion 120 is provided between the first end 101 and the second end 102. The fall-off prevention portion 120 is close to the first end 101. The outer diameter of the fall-off prevention portion 120 is smaller than the outer diameter of the second end 102. When in use, the cavity opening clamps the fall-off prevention portion 120 to prevent falling off.

As shown in FIG. 4, the fall-off prevention portion 120 may be formed by inclining inward along the axis of the flexible shell 100 from the second end 102 to the first end 101; the flexible shell 100 may be provided with a tapered portion tapering from the second end 102 to the first end 101; or a circle of fall-off prevention grooves is provided between the first end 101 and the second end 102.

In order to provide a certain guide for the inflation of the flexible shell 100 to prevent the flexible shell 100 from being in an irregular shape when inflated, as shown in FIG. 3 to FIG. 4, in an embodiment, the flexible shell 100 is provided with an electrode sheet. The thickness of the part provided with the electrode sheet 200 of the flexible shell 100 is greater than the thickness of the corrugated portion 110, that is, the thickness of the corrugated portion 110 is smaller than the part of the flexible shell 100 provided with the electrode sheet 200, so that the corrugated portion 110 is easy to be inflated.

As shown in FIG. 4, in an embodiment, the fall-off prevention portion 120 includes an extending portion 121 and a clamping portion 122. The extending portion 121 is close to the electrode sheet 200 relative to the clamping portion 122. The thickness of the fall-off prevention portion 120 decreases from the extending portion 121 to the clamping portion 122, so that the flexible shell 100 may expand evenly when inflated, and the treatment area of the treatment tip 10 (the area where the electrode sheet 200 is located) forms a cylinder with a uniform outer diameter to fit the cavity surface. If the fall-off prevention portion 120 is too thick, it will disturb the expansion of the end of the fall-off prevention portion (the first end 101) of the flexible shell 100, thereby forming an inverted tapered cylinder. As shown in FIG. 4, in an embodiment, the thickness of the first end 101 of the flexible shell 100 increases from the clamping portion 122 to the first end 101, so that the first end 101 maintains its original shape without deformation when inflated to prevent installation member 500 from coming out of port 100b.

In order to facilitate the deformation of the flexible shell 100, in an embodiment, the thickness of the clamping portion 122 may be the same as the thickness of the corrugated portion 110. As shown in FIG. 4, in an embodiment, the flexible shell 100 provided with the electrode sheet 200 part has a uniform thickness, so as to further enable the flexible shell 100 to be inflated and expanded evenly.

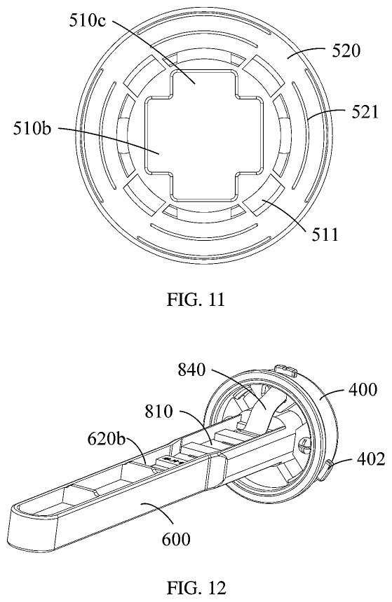

Further, in order to prevent the installation member 500 from falling off the flexible shell 100, as shown in FIG. 10 and FIG. 11, in an embodiment, the ring surface of the thimble 520 is provided with a strip protrusion 521 extending circumferentially along the extension barrel 510.

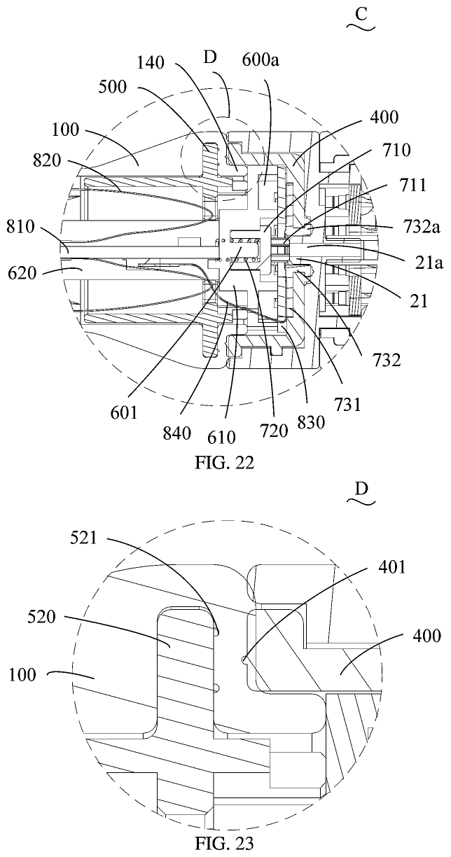

As shown in FIG. 22 to FIG. 23, the strip protrusion 521 is provided on the side of the thimble 520 facing the opening of the port 100b. By setting the strip protrusion 521 on the annular surface of the thimble 520, the flexible shell 100 presses the strip protrusion 521 to make the strip protrusion 521 deformed, so that the strip protrusion 521 is embedded in the groove wall of the embedded groove. When the installation member 500 is subjected to outward pulling force, the strip protrusion 521 contacts the flexible shell 100, which increases the friction between the installation member 500 and the flexible shell 100, so that the thimble 520 does not easily detach. In this way, it improves the stability of the connection between the installation member 500 and the flexible shell 100.

As shown in FIG. 10 and FIG. 11, there may be a plurality of strip protrusions. The plurality of strip protrusions 521 are provided at circumferential intervals around the extension barrel 510, and/or the plurality of strip protrusions 521 are provided radially on the thimble 520, thereby further improving the stability of the connection between the installation member 500 and the flexible shell 100. Different from the above embodiment, as shown in FIG. 26 to FIG. 28, in an embodiment, the ring surface of the thimble 520 is not provided with a strip protrusion 521.

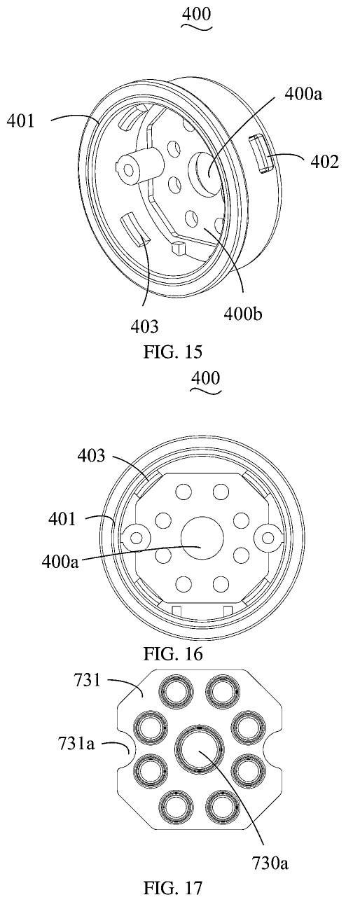

As shown in FIG. 15, FIG. 16 and FIG. 23, the side of the installation seat 400 facing the flexible shell 100 may be provided with an annular protrusion 401. When the installation seat 400 is installed on the flexible shell 100, the flexible shell 100 may apply a force to deform the annular protrusion 401, so that the annular protrusion 401 is embedded in the flexible shell 100. Moreover, the installation seat 400 and the installation member 500 snap together, and the annular protrusion 401 contacts the flexible shell 100, which thereby make the flexible shell 100 clamped between the installation member 500 and the installation seat 400. The annular protrusion 401 enhances the effect of clamping and sealing.

Since the thimble 520 is protruding from the wall of the extension barrel 510, in order to improve the strength of the thimble 520, as shown in FIG. 10, in an embodiment, the installation member 500 is provided with a stiffener 530, and the stiffener 530 is located on the outer wall of extension barrel 510, and connects the thimble 520.

The stiffener 530 is located on the side of the thimble 520 facing away from the port 100b. The stiffener 530 connects the extension barrel 510 and the thimble 520 to prevent the thimble 520 from easily breaking after collision, thereby improving the strength of the thimble 520 and thus improving the service life of the installation member 500. As shown in FIG. 10, there may be a plurality of stiffeners 530. The plurality of stiffeners 530 are provided at intervals in the circumferential direction of the extension barrel 510 to improve the strength of the thimble 520 as a whole.

As shown in FIG. 10, in an embodiment, the plurality of stiffeners 530 are provided at non-equidistant intervals in the circumferential direction of the extension barrel 510. As shown in FIG. 3, the inner wall of the first end 101 of the flexible shell 100 is provided with a positioning block 130. A positioning groove is formed between the two stiffeners 530. Since the plurality of stiffeners 530 are not equidistant in the extension barrel 510, the positioning block 130 cooperates with the positioning groove to prevent the installation member 500 from fooling, so as to limit the installation position of the support component 600 in the flexible shell 100.

As shown in FIG. 26, in an embodiment, the inner wall surface of the extension barrel 510 is provided with a guide rig 540. The guide rig 540 extends along the length direction of the extension barrel 510. On the one hand, it guide the support component 600 inserted into the fool-proofing port 510a to make the support component 600 slide along the guide rig 540; on the other hand, the shaking of the support component 600 on the installation member 500 is reduced, and the structural strength of the installation member 500 is enhanced.

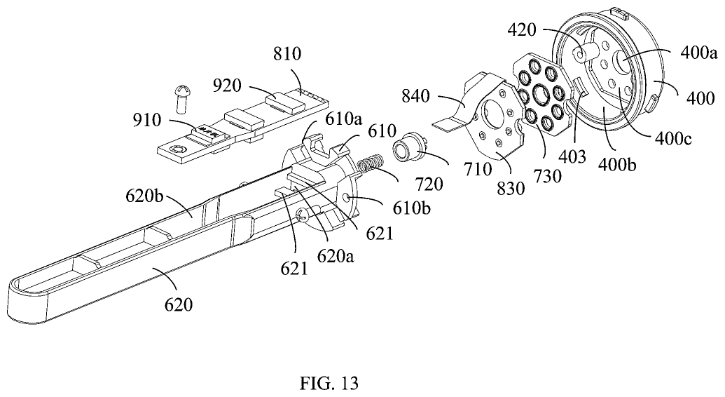

As shown in FIG. 3 and FIG. 12, in an embodiment, the support component 600 of the treatment tip 10 is used to support the flexible shell 100 so that the flexible shell 100 may maintain a certain shape and be smoothly inserted into the cavity. As shown in FIG. 12, FIG. 13 and FIG. 29, the support component 600 is provided in a strip shape to better support the flexible shell 100. As shown in FIG. 4 and FIG. 10, the extension barrel 510 is provided with a fool-proofing port 510a connected with the inflatable cavity 100a. The fool-proofing port 510a is used for the support component 600 to pass through. By arranging the fool-proofing port 510a on the extension barrel 510, the support component 600 enters the inflatable cavity 100a from the fool-proofing port 510a in a certain direction, thereby preventing the support component 600 from being installed backwards, causing workers to rework or affecting product quality.

That is to say, through the stiffener 530 on the extension barrel 510 and the positioning block 130 on the inner wall of the flexible shell 100, the installation member 500 is installed to prevent fooling. In addition, the installation member 500 is provided with a fool-proofing port 510a, which limits the extending position of the support component 600 so that the support component 600 is installed in the flexible shell 100 at a certain angle.

Further, as shown in FIG. 10 and FIG. 11, in an embodiment, the fool-proofing port 510a is a cross groove, and the fool-proofing port 510a includes a first groove port 510b and a second groove port 510c intersecting with each other. The width of the first groove port 510b is greater than the width of the second groove port 510c.

As shown in FIG. 11, the outer diameter of the support component 600 is smaller than the width of the first groove port 510b and larger than the width of the second groove port 510c, so that when the treatment tip 10 is assembled, the support component 600 may enter the inflatable cavity 100a from the first groove port 510b to position the support component 600. When the support component 600 try to enter the inflatable cavity from the second groove port 510c, because the outer diameter of the support component 600 is greater than the width of the second groove port 510c, the support component 600 may not pass through the second groove port 510c. The worker may find the installation is reversed and the direction of the support component 600 may be adjusted in time to avoid subsequent rework and improve the efficiency of product assembly.

The installation member 500 and the installation seat 400 may be connected in various ways, which may be detachable connection or non-detachable connection. As shown in FIG. 4 and FIG. 12, the installation member 500 and the installation seat 400 are detachably connected, thereby simplifying the production process and facilitating manufacturing workers' assembly operations.

In an embodiment, the installation member 500 is engaged with the installation seat 400. Specifically, as shown in FIG. 10 and FIG. 11, in one embodiment, the extension barrel 510 is provided with a hook piece 511 at one end close to the thimble 520, and the hook piece 511 is used to snap with the installation seat 400. One end of the extension barrel 510 located at the port 100b protrudes outward to form a hook portion 511a. The hook piece 511 is engaged with the installation seat 400 to realize the connection between the installation member 500 and the installation seat 400. In addition, as shown in FIG. 22 to FIG. 23, when the installation member 500 is clamped with the installation seat 400, the thimble 520 and the installation seat 400 clamp the end of the first end 101 of the flexible shell 100, thereby achieving a sealing effect. The liquid is prevented from entering the flexible shell 100 from the gap between the end of the first end 101 and the installation seat 400.

As shown in FIG. 12 and FIG. 13, the installation seat 400 is provided with a buckle piece 403 that cooperates with the hook piece 511. The hook piece 511 may be of various types. By engaging the hook piece 511 with the buckle piece 403, the installation member 500 and the installation seat 400 are quickly connected to improve assembly efficiency.

The buckle piece 403 may be located on the outer wall of the installation seat 400 or on the inner wall of the installation seat 400. As shown in FIG. 13, FIG. 15 and FIG. 16, the installation seat 400 is provided with a receiving groove 400b at one end facing the flexible shell 100, and the buckle piece 403 is located in the groove wall of the receiving groove 400b.

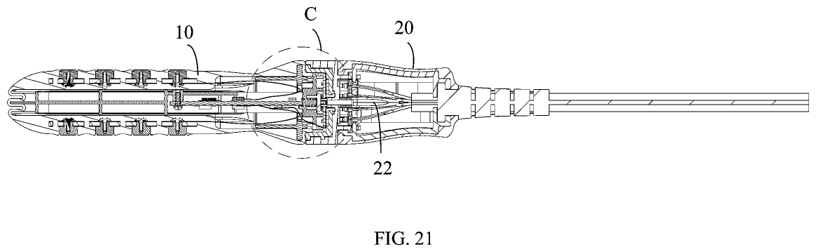

As shown in FIGS. 21 and 22, the first end 101 of the flexible shell 100 may extend into the receiving groove 400b to protect the installation member 500 from breaking at the connection between the installation member 500 and the installation seat 400, thereby improving the stability of the connection between the installation member 500 and the installation seat 400. The flexible shell 100 is installed on the installation member 500 by engaging the hook piece 511 of the installation member 500 with the buckle piece 403 of the wall of the receiving groove 400b.

As shown in FIG. 15, the installation seat 400 is provided with an inflation hole 400a that communicates with the inflatable cavity 100a, so that after the installation seat 400 is connected to the handle 20, the air pump in the handle 20 may inflate the flexible shell 100 through the inflation hole 400a to treat the user.

Since the treatment tip 10 needs to be cleaned after the treatment course, during cleaning, the treatment tip 10 is detached from the handle 20. In order to prevent the liquid from entering the inflation cavity 100a from the inflation hole 400a of the installation seat 400 during the cleaning process. In an embodiment, as shown in FIG. 13 and FIG. 22, the treatment tip 10 further includes a sealing gasket 730, which is installed inside the installation seat 400. The treatment tip 10 further includes a sealing member 710 that is elastically abutted against the sealing gasket 730 to block the inflation hole 400a.

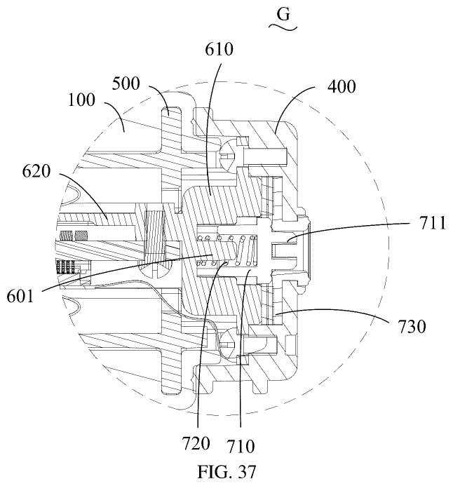

As shown in FIG. 22, FIG. 36 to FIG. 37, the sealing gasket 730 is located on the side of the installation seat 400 away from the handle 20. The sealing gasket 730 is tightly attached to the inner wall of the installation seat 400 to prevent liquid from flowing between the installation seat 400 and the sealing gasket sealing gasket 730 to enter. The sealing member 710 elastically abuts the sealing gasket 730. The outer diameter of the sealing member 710 is larger than the diameter of the inflation hole 400a. Before the treatment tip 10 is installed on the handle 20, the sealing member 710 blocks. the inflation hole 400a in an elastic force, and the inflatable cavity 100a is isolated from the external environment. On the one hand, it prevents the liquid from entering the inflatable cavity 100a during the cleaning process of the treatment tip 10 and avoids short circuit of the internal circuit structure. On the other hand, when the treatment tip 10 is inflated, the sealing member 710 and the sealing gasket 730 cooperate to prevent air leakage from the treatment tip 10 and improve the air tightness of the treatment tip 10.

In this regard, as shown in FIG. 4, FIG. 16 and FIG. 17, in an embodiment, the sealing gasket 730 is provided with a bleed hole 730a. The bleed hole 730a is opposite to the inflation hole 400a, and the gas passes through the inflation hole 400a and the bleed hole 730a to enter the inflatable cavity 100a. As shown in FIG. 13, FIG. 16 and FIG. 17, the aperture of the bleed hole 730a is smaller than the aperture of the inflation hole 400a. The outer diameter of the sealing member 710 may be larger than the aperture of the bleed hole 730a, so that the sealing member 710 elastically contacts the sealing gasket 730. The sealing member 710 may seal the bleed hole 730a, and then seal the inflation hole 400a, thereby achieving the sealing of the treatment tip 10 and improving the sealing of the product. As shown in FIG. 22, when the sealing member 710 is subjected to an external force, the sealing member 710 moves away from the inflation hole 400a, thereby opening the inflation hole 400a to deliver gas into the inflatable cavity 100a. The sealing member 710 is located on the side of the installation seat 400 facing away from the handle 20. Through the elastic contact between the sealing member 710 and the installation seat 400, the sealing of the inflation hole 400a is achieved to prevent small foreign matter from entering the inflatable cavity 100a, thereby improving the service life of the components in the inflatable cavity 100a.

There are many ways to achieve elastic contact between the sealing member 710 and the installation seat 400. Specifically, as shown in FIG. 12 to FIG. 13 and FIG. 22. In an embodiment, the treatment tip 10 also includes an elastic member 720, the elastic member 720 is located between the sealing member 710 and the support component 600, so that the sealing member 710 is in contact with the edge of the inflation port.

As shown in FIG. 22, the sealing member 710 may be connected to the support component 600 through the elastic member 720. The elastic member 720 is located between the sealing member 710 and the support component 600. When the support component 600 is installed on the installation seat 400, the elastic member 720 is in a compressed state and has the thrust to restore the deformation, so that the sealing member 710 is abutted against the periphery of the inflation hole 400a, and the inflation hole 400a is seal.

In order to facilitate the installation of the elastic member 720 and improve the contact reliability of the sealing member 710, as shown in FIG. 21 to FIG. 22, in one embodiment, the support component 600 is provided with a post 601 at one end facing the inflation hole 400a. The elastic member 720 is sleeved on the outer periphery of the post 601, and the sealing member 710 is sleeved on the outer periphery of the elastic member 720.

As shown in FIG. 22, the base 610 of the support component 600 is provided with a post 601. The post 601 extends in the direction of the inflation hole 400a. By placing the elastic member 720 on the outer periphery of the post 601, the elastic member 720 is positioned to prevent the sealing member 710 on the elastic member 720 from deviating from the inflation hole 400a and failing to seal the inflation hole 400a. The sealing member 710 may be provided in a cylindrical shape. The sealing member 710 is slotted at one end towards the post 601. The sealing member 710 is sleeved on the outer circumference of the elastic member 720 for installing the sealing member 710. It may be understood that the elastic member 720 may be a spring.

As shown in FIG. 22 and FIG. 30, in an embodiment, a side of the base 610 facing the inflation hole 400a is recessed to form an accommodation groove 600a, the post 601 is provided at the bottom of the accommodation groove 600a, and the accommodation groove 600a is provided with a gas hole 600b that communicates with the inflatable cavity 100a.

As shown in FIG. 22 and FIG. 30, by setting an accommodation groove 600a on the base 610 of the support component 600, the volume of the base 610 will not be too large, which facilitates the connection between the installation member 500 and the installation seat 400. At the same time, the length of the treatment tip 10 will not be too large. The inflation hole 400a may be provided at the bottom of the accommodation groove 600a. When the sealing member 710 is moved away from the inflation hole 400a, the gas enters the accommodation groove 600a from the inflation hole 400a and passes through the gas hole 600b of the accommodation groove 600a to reach the inflatable cavity 100a.

The sealing member 710 rebounds to the installation seat 400, when the support component 600 is installed with the installation seat 400, the sealing member 710 may deviate from the inflation hole 400a. For this, as shown in FIG. 20 and FIG. 22, in one embodiment, the sealing member 710 is provided with a positioning post 711 extending into the inflation hole 400a.

As shown in FIG. 21 and FIG. 22, by providing the positioning post 711 on the sealing member 710, during installation, the sealing member 710 may be installed on the support component 600 first, and then the positioning post 711 may be aligned with the inflation hole 400a, and the support component 600 may be installed in conjunction with the installation seat 400. The positioning group plays a positioning role, so that when the support component 600 and the installation seat 400 are installed, there is no need to consider the cooperation between the sealing member 710 on the support component 600 and the installation seat 400, thereby improving the assembly efficiency of the treatment tip 10. In addition, during the rebound process of the sealing member 710, the positioning post 711 plays a guiding and positioning role, so that the sealing member 710 may successfully reset and seal the inflation hole 400a.

As shown in FIG. 20 and FIG. 22, there are plurality of positioning posts 711, and a gap is provided between two positioning posts 711. The gas may flow out from the gap between the positioning posts 711 and enter the accommodation groove 600a of the base 610 of the support component 600. As shown in FIG. 20, a plurality of positioning posts 711 may be provided at axial intervals around the sealing member 710. The ends of the positioning posts 711 are provided to bevel so that the positioning posts 711 may be inserted into the inflation hole 400a of the installation seat 400.

In order to improve the airtightness of the treatment tip 10 and reduce air and water leakage, as shown in FIGS. 13, 17 and 22, in one embodiment, the treatment tip 10 also includes a sealing gasket 730. The sealing gasket 730 includes a gasket body 731, the bleed hole 730a is opened in the gasket body 731, and the gasket body 731 is pressed between the support component 600 and the installation seat 400. The gasket body 731 is provided with a sealing ring 732, the sealing ring 732 extends from the bleed hole 730a along the hole wall of the inflation hole 400a, and protrudes from the end face of installation seat 400.

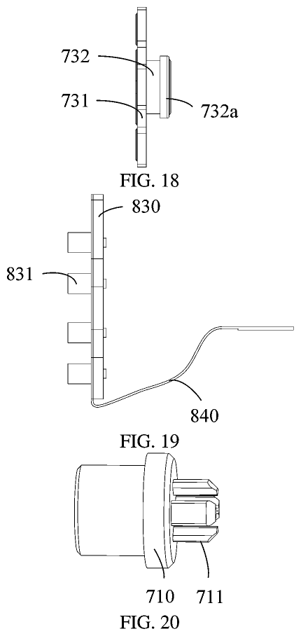

As shown in FIG. 13, FIG. 18 and FIG. 22, by installing the support component 600 on the installation seat 400, the gasket body 731 may be installed on the inner wall of the installation seat 400. The gasket body 731 is provided with a sealing ring 732. The sealing ring 732 extends along the wall surface of the inflation hole 400a. Gas may enter the receiving groove 400b of the support component 600 along the sealing gasket 730 to prevent gas or liquid from entering between the sealing ring 732 and the installation seat 400, thereby improving the airtightness of the product.

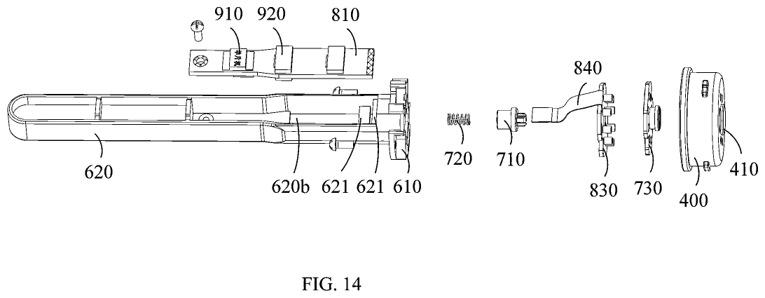

As shown in FIG. 14 and FIG. 22, in one embodiment, the installation seat 400 is provided with a flange 410 on the periphery of the inflation hole 400a, and the flange 410 is located away from the installation seat 400. On one side of the support component 600, the sealing ring 732 is provided with an outer flange 732a, and the outer flange 732a is embedded in and exposed on the flange 410.

As shown in FIG. 14, the outer flange 732a is embedded in the flange 410 of the installation seat 400 and is exposed at the end of the installation seat 400. On the one hand, it facilitates the connection of the sealing gasket 730 and the installation seat 400. The sealing gasket 730 may be clamped on the installation seat 400 to fix the sealing gasket 730; on the other hand, after the installation seat 400 is connected to the handle 20, the end of the sealing ring 732 is in contact with the groove bottom of the installation groove 20a of the handle 20 for sealing to prevent the gas from flowing out of the gap between the gas installation seat 400 and the handle 20, thus improving the anti-leakage performance of the product.

As shown in FIG. 16 to FIG. 17 and FIG. 22, in one embodiment, the installation seat 400 is provided with a sealing groove 400c that accommodates the gasket body 731. The sealing groove 400c may be adapted to the gasket body 731. By setting the sealing groove 400c at the bottom of the receiving groove 400b, the gasket body 731 is installed in the sealing groove 400c, thereby avoiding the sealing gasket 730 dislocated on the installation seat 400, which improves the sealing performance of the product. It should be noted that the shape of the gasket body 731 may be a quadrilateral, a pentagon or other regular or irregular shapes. The sealing groove 400c limits the gasket body 731 to prevent the gasket body 731 from rotating in the receiving groove 400b. As shown in FIG. 17, in one embodiment, the gasket body 731 is also provided with an escaping port 731a to avoid the stud 420 to further position the gasket body 731 and prevent the gasket body 731 from rotating.

In one embodiment, the treatment tip 10 has a four-level waterproof structure.

Specifically, as shown in FIG. 3, FIG. 4 and FIG. 22, an extension ring 140 is provided at the port of the flexible shell 100. The extension ring 140 extends into the receiving groove 400b of the installation seat 400, thereby forming a first-level waterproof structure which makes it difficult for liquid to penetrate between the flexible shell 100 and the installation seat 400. As shown in FIG. 15, FIG. 22 to FIG. 23, the installation seat 400 is provided with an annular protrusion 401. When the installation seat 400 is installed on the flexible shell 100, the flexible shell 100 squeezes and deforms the annular protrusion 401. The annular protrusion 401 is embedded in the flexible shell 100, thereby forming a second-level waterproof structure, so that liquid may not easily enter between the flexible shell 100 and the installation seat 400.

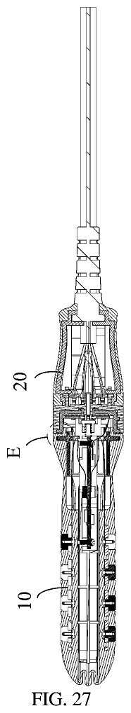

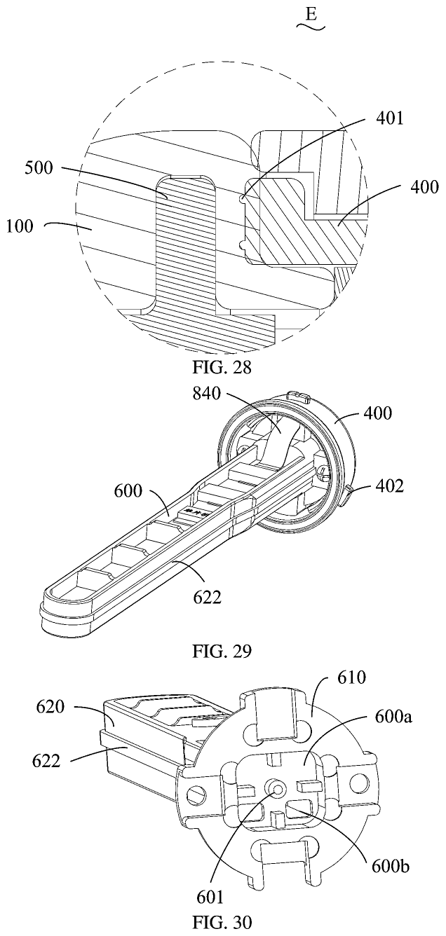

It should be noted that, as shown in FIGS. 27 and 28, in one embodiment, the installation seat 400 has plurality of annular protrusions 401, and the plurality of annular protrusions 401 are spaced apart along the width direction of the installation seat to enhance the waterproofing effect.

As shown in FIG. 20 and FIG. 22, the sealing member 710 elastically abuts the periphery of the inflation hole 400a to seal the inflation hole 400a, thus forming a third-level waterproof structure to prevent liquid from entering the treatment tip 10 from the inflation hole 400a. As shown in FIG. 18 and FIG. 22, the sealing gasket 730 is installed on the installation seat 400. The sealing gasket 730 includes a gasket body 731 and a sealing ring 732. The gasket body 731 cover on the periphery of the inflation hole. The sealing ring 732 extends along the hole wall of the inflation hole 400a. The sealing ring 732 is provided with an outer flange 732a exposed at the end of the installation seat 400 to prevent liquid from flowing from the installation seat 400 and the handle 20 to the treatment tip 10, thereby forming a fourth-level waterproof structure.

As shown in FIG. 13, in one embodiment, the support component 600 includes a base 610 and a support portion 620 installed on the base 610. The support portion 620 extends into the inflatable cavity 100a of the flexible shell 100. The flexible shell 100 provides support to facilitate the insertion of the treatment tip 10 into the cavity.

As shown in FIG. 21 and FIG. 22, the base 610 of the support component 600 may be installed on the installation seat 400, thereby positioning the support component 600 and preventing the support component 600 from shaking in the inflatable cavity 100a of the flexible shell 100, and affecting the use of treatment tip 10. At the same time, the installation member 500 passes through the base 610 and is connected to the installation seat 400 to realize the connection between the flexible shell 100 and the installation seat 400.

As shown in FIG. 12 to FIG. 14, in one embodiment, the end of the support portion 620 is provided in an arc to match the shape of the treatment tip 10. The support portion 620 is not easy to damage the flexible shell 100 when squeezed. As shown in FIG. 12 and FIG. 13, in one embodiment, the outer diameter of an end of the support portion 620 is smaller than the outer diameter of the end of the support portion 620 close to the base 610. That is to say, the size of the end of the support portion 620 close to the base 610 is larger than the size of the end of the support portion 620 away from the base 610. On the one hand, it leaves space for the corrugated portion 110 to escape and facilitates assembly; on the other hand, the end of the portion 620 close to the base 610 may have a large space to install the adapter plate 810 to meet actual needs.

It should be noted that the support portion 620 and the base 610 may be integrally formed to improve the structural stability of the support component 600. As shown in FIG. 13, the support component 600 is provided with an avoidance port 610a of the hook piece 511. The avoidance port 610a may be an opening. As shown in FIG. 29 and FIG. 30, in one embodiment, a reinforcement bar 622 is provided on the side wall of the support portion 620, and the reinforcement bar 622 extends along the circumferential direction of the support portion 620.

As shown in FIG. 12 to FIG. 13, in one embodiment, the base 610 of the support component 600 is installed on the installation seat 400, thereby fixing the support component 600 to the installation seat 400 and further preventing the support component 600 from swaying in the inflatable cavity 100a of the flexible shell 100, which is convenient for daily treatment.

As shown in FIG. 12, in an embodiment, the base 610 is screwed to the installation seat 400. The base 610 of the support component 600 is screwed to the installation seat 400. When the support component 600 or the installation seat 400 is damaged, the damaged parts may be replaced without replacing the entire treatment tip 10, thereby avoiding material waste.

Specifically, as shown in FIG. 12 to FIG. 13, in one embodiment, the installation seat 400 is provided with a stud 420, and the base 610 is provided with an installation hole 610b for threaded connectors to pass through. A stud 420 may be set at the bottom of the receiving groove 400b of the installation seat 400, and the screw connector passes through the installation hole 610b on the base 610 to connect to the stud 420, so that the support component 600 is installed on the installation seat 400, the installation operation of the support component 600 is implemented.

As shown in FIG. 12 and FIG. 21, the treatment tip 10 also includes an adapter plate 810. The adapter plate 810 is installed in the inflatable cavity 100a. The hard circuit board 300 is electrically connected to the handle 20 through the adapter plate 810. By setting the adapter plate 810, the hard circuit board 300 may be electrically connected to the adapter plate 810 first, and then the adapter plate 810 may be electrically connected to the handle 20, in this way, it has no need for each wire of the hard circuit board 300 passes through the port 100b of the flexible shell 100 and is electrically connected to the handle 20. In this way, the penetration of wires is reduced and the amount of wires is saved.

As shown in FIG. 4, FIG. 12 and FIG. 21, the hard circuit board 300 is electrically connected to the adapter plate 810 through the first flexible circuit board 820, thereby completing the connection between the signal line and the adapter plate 810, which further facilitates the connection between the signal line and the adapter plate 810, and improves the assembly efficiency of the treatment tip 10.

In order to further facilitate the connection between the first flexible circuit board 820 and the adapter plate 810, as shown in FIG. 13, in one embodiment, the adapter plate 810 is provided with a cable connection end 920. The first flexible circuit board 820 may be plugged into the cable connection end 920 to realize the quick connection between the first flexible circuit board 820 and the adapter plate 810, thereby improving the connection efficiency between the hard circuit board 300 and the adapter plate 810.

As shown in FIG. 13 and FIG. 22, the treatment tip 10 may also include a main board 830. The main board 830 may be clamped between the support component 600 and the sealing gasket 730 to fix the main board 830. The main board main board 830 is used for electrical connection with the hard circuit board 300. As shown in FIG. 14 and FIG. 19, the main board main board 830 is also provided with a connection terminal 831, and the handle 20 is provided with a plug end for electrically connection with the connection terminal 831. The treatment tip 10 may be electrically connected to the handle 20, so that the handle 20 may read and display the data of the treatment tip 10, such as usage time, usage duration, etc. The connection terminal 831 passes through the installation seat 400 and is exposed on the end surface of the installation seat 400, so that after the installation seat 400 is connected to the handle 20, the connection terminal 831 is connected to the plug end on the handle 20.

As shown in FIGS. 21 and 22, The adapter plate 810 is electrically connected to the main board 830. The main board 830 is used to electrically connect to the handle 20, thereby realizing the electrical connection between the adapter plate 810 and the handle 20. As shown in FIG. 12, the adapter plate 810 is installed on the support component 600 to reduce the distance between the adapter plate 810 and the main board 830.

As shown in FIG. 12, in one embodiment, the treatment tip 10 also includes a second flexible circuit board 840 installed on the main board 830. The second flexible circuit board 840 is used to electrically connected with the adapter plate 810, thereby reducing the use of wires and facilitating the assembly of the treatment tip 10. It should be noted that there may be a plurality of cable connection ends 920 on the adapter plate 810. The cable connection end 920 may be connected to the first flexible circuit board 820 or to the second flexible circuit board 840, thereby facilitating the connection between the first flexible circuit board 820, the second flexible circuit board 840 and the adapter plate 810.

In order to prevent the adapter plate 810 from being squeezed by external force and causing damage to the adapter plate 810, as shown in FIG. 12, FIG. 21 and FIG. 22. In one embodiment, the adapter plate 810 is installed on a side of the support component 600 toward the hard circuit board 300. Since the hard circuit board 300 and the electrode sheet 200 have a certain hardness, when the flexible shell 100 is squeezed by an external force, the hard circuit board 300 and the electrode sheet 200 will not dent downwards, which protects the plate adapter plate 810 and increases the service life of the product. Specifically, as shown in FIG. 21 and FIG. 22, the adapter plate 810 is installed on the side of the support portion 620 facing the hard circuit board 300.

There are many ways to install the adapter plate 810 on the support component 600. In order to improve the assembly efficiency, as shown in FIG. 13 to 14, in one embodiment, one end of the adapter plate 810 is inserted into the support component 600, and the other end is screwed to the support portion 620.

As shown in FIG. 13, during assembly, one end of the adapter plate 810 is inserted into the support portion 620, and the other end of the adapter plate 810 is screwed to the support portion 620 through screws. This not only firmly installs the adapter plate 810 on the support portion 620 also reduces the use of screws, reduces the assembly process of workers, and improves the assembly efficiency of the product.

The socket 620a may be formed in a variety of ways, as shown in FIG. 13 to FIG. 14, in one embodiment, the support portion 620 is provided with two stopper bars 621. A socket 620a is formed between two stopper bars 621. The two stopper bars 621 are offset in the vertical direction.

As shown in FIG. 13, the two stopper bars 621 are not in the same vertical direction. The space between the two stopper bars 621 is larger, which is convenient for the insertion of the adapter plate 810, thus improving the assembly efficiency of plate 810. At the same time, the stopper bar 621 may guide the adapter plate 810 into the socket 620a, further facilitating the insertion of the adapter plate 810.

As shown in FIG. 12 to FIG. 13, in one embodiment, the support portion 620 is provided with a profiled slot 620b, and the profiled slot 620b is adapted to the adapter plate 810. On the one hand, the profiled slot 620b has the anti-fool function to prevent the adapter plate 810 from being installed upside down, causing rework, thereby reducing the possibility of an incorrect installation, and improving the consistency of the product; on the other hand, it reduces the weight of the support portion 620 and reduces the overall weight of the product, thereby facilitating usage. As shown in FIG. 13, the two ends of the stopper bar 621 are respectively connected to the groove walls on opposite sides of the profiled slot 620b. The two stopper bars 621 not only form the socket 620a, but also increase the strength of the profiled slot 620b, thereby reducing the possibility of deformation of profiled slot 620b. The bottom of the profiled slot 620b may be provided with an installation opening so that the first flexible circuit board 820 installed on the hard circuit board 300 may pass through the installation opening to realize the connection between the first flexible circuit board 820 and the adapter plate 810. In addition, because the adapter plate 810 is installed in the profiled slot 620b, the strength of the support portion 620 may be improved to prevent the support portion 620 from being easily broken.

The adapter plate 810 has various shapes. As shown in FIG. 12 to FIG. 13, in one embodiment, the adapter plate 810 extends along the length direction of the flexible shell 100, making full use of the inflatable cavity 100a of the flexible shell 100, thereby preventing the flexible shell 100 from increasing radially and reducing the volume of the flexible shell 100. As shown in FIG. 12, the adapter plate 810 may be installed at a position of the support portion 620 close to the base 610, thereby reducing the distance between the adapter plate 810 and the main board main board 830, and reducing the length of the second flexible circuit board 840 and reducing costs.

As shown in FIG. 13 and FIG. 14, the width of the end of the adapter plate 810 close to the port 100b is greater than the width of the end of the adapter plate 810 away from the port 100b. By setting the widths of the two ends of the adapter plate 810 to be different, it prevents fooling. In addition, the outer diameter of the end of the support portion 620 away from the base 610 may be smaller than the outer diameter of the end of the support portion 620 close to the base 610, which facilitate the insertion of the support portion 620 into the inflatable cavity 100a from the port 100b, thereby facilitating assembly. At the same time, the outer diameter of the second end 102 of the flexible shell 100 may be smaller than the outer diameter of the first end 101 to facilitate inserting the flexible shell 100 into the vaginal cavity.

As shown in FIG. 13 to FIG. 14, since the cable connection end 920 is a standard part and is large in size, the cable connection end may be installed on the end of the adapter plate 810 close to the base 610, in this way, the cable connection end 920 is reasonably installed on the adapter plate 810. As shown in FIG. 13. The adapter plate 810 is also provided with a control unit 910. The control unit 910 is used for signal conversion. It may convert the signal of the hard circuit board 300 into a digital signal. The digital signal is passed to the main board 830, so that the usage of the treatment tip 10 may be recorded, such as the specific usage time, usage duration, etc. The control unit 910 is small in size. As shown in FIG. 13, the control unit 910 may be installed on the smaller end of the adapter plate 810.

As shown in FIG. 1, the installation seat 400 and the handle 20 are detachably connected, and the treatment tip 10 may be replaced. The handle 20 may be fully utilized to avoid the waste of materials and save the user's treatment costs. As shown in FIG. 1, In one embodiment, the installation seat 400 is engaged with the handle 20. Through the snap connection between the installation seat 400 and the handle 20, the installation seat 400 and the handle 20 may be connected quickly and efficiently. The operation is simple and convenient, and it is convenient for the user's assembly operation.

Further, as shown in FIG. 1 to FIG. 2, in one embodiment, one of the installation seat 400 and the handle 20 is provided with a snap block 402, and the other is provided with a snap slot 20b. The snap block 402 is snapped into the snap slot 20b to realize the connection between the installation seat 400 and the handle 20.

As shown in FIG. 1 to FIG. 2, in one embodiment, the outer wall of the installation seat 400 is provided with a snap block 402, and the handle 20 is provided with a snap slot 20b. The handle 20 is provided with an installation groove 20a. The installation groove 20a is used to accommodate the installation seat 400. The groove wall of the installation groove 20a is provided with a snap slot 20b. through the cooperation of the snap block 402 and the snap slot 20b, the flexible shell 100 and the handle 20 are connected together.

In order to facilitate the user's operation, as shown in FIG. 1, in one embodiment, the snap slot 20b includes a channel slot 20c available for the snap block 402 extending into and a clamping slot 20d extending circumferentially along the handle 20. The channel slot 20c is communicated with the clamping slot 20d.

As shown in FIG. 1 to FIG. 2, the snap block 402 enters the snap slot 20b through the channel slot 20c. The installation seat 400 is rotatable so that the snap block 402 moves in the clamping slot 20d. The clamping slot 20d clamps the snap block 402 to prevent the buckle from escaping from the clamping slot 20d. Since the flexible shell 100 is installed on the installation seat 400, the connection between the flexible shell 100 and the handle 20 is realized. The method of removing the flexible shell 100 is opposite to the above. First, the flexible shell 100 is rotated, and the snap block 402 of the installation seat 400 moves in the clamping slot 20d, and exits the snap slot 20b through the channel slot 20c, and then the flexible shell 100 may be quickly separated from the handle 20. As shown in FIG. 1, the snap slot 20b structure not only facilitates the installation and disassembly of the flexible shell 100 and the handle 20, but also improves the stability of the connection between the flexible shell 100 and the handle 20.

As shown in FIG. 21 and FIG. 22, the handle 20 is provided with an abutting pillar 21 corresponding to the inflation hole 400a. When the handle 20 is connected to the installation seat 400, the abutting pillar 21 extends into the inflation hole 400a and abuts with the sealing member 710, so that the sealing member 710 is separated from the inflation hole 400a, and the inflation hole 400a is opened. As shown in FIG. 21 and FIG. 22, in one embodiment, a gas delivery port 21a may be provided on the abutting pillar 21 of the handle 20, and the gas delivery port 21a may be provided with the end of the abutting pillar 21. The abutting pillar 21 is communicated to the gas pump through the gas tube 22, and the gas tube 22 is communicated with the gas delivery port 21a, so that the gas from the gas tube 22 enters into the accommodation groove 600a through the gas delivery port 21a, and then from the accommodation groove 600a to the inflatable cavity 100a of the flexible shell 100.

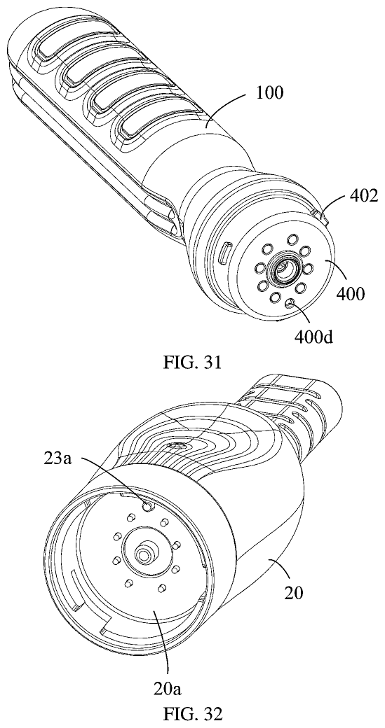

As shown in FIG. 31 to FIG. 32, in one embodiment, the handle 20 is provided with a limiting pillar 23a that may extend into and out of the treatment tip 10, and the installation seat 400 is provided with a limiting hole 400d to accommodate the limiting pillar 23a. The limiting hole 400d is offset from the axis of the treatment tip 10. When it is necessary to remove the treatment tip 10, users may first move the limiting pillar 23a towards the inside of the handle 20, move the limiting pillar 23a out of the limiting hole 400d, then rotate the treatment tip 10. The snap block 402 moves in the snap slot 20b, and finally the snap block 402 leaves the snap slot 20b, thereby realizing the separation of the treatment tip 10 and the handle 20. When the handle 20 needs to be connected to the treatment tip 10, the treatment tip 10 may be rotated, and the limiting pillar 23a may be inserted into the limiting hole 400d, which means that the handle 20 and the treatment tip 10 have been matched.

When the handle 20 and the treatment tip 10 are assembled, since the limiting pillar 23a extends into the limiting hole 400d, and the position of the limiting hole 400d on the installation seat 400 deviates from the axis of the treatment tip 10, the limiting pillar 23a hinders the rotation of the treatment tip 10, making it impossible to rotate the treatment tip 10. It should be noted that when the limiting pillar 23a exits the limiting hole 400d, the end of the limiting pillar 23a may be lower than the groove wall of the installation groove 20a, or the limiting pillar 23a may partially protrude out of the wall surface of the installation groove 20a, as long as it does not affect the rotation of the treatment tip 10. The limiting pillar 23a may be located at the bottom of the installation groove 20a, or may be located at the side wall of the installation groove 20a, specifically it may be set according to the actual situation.

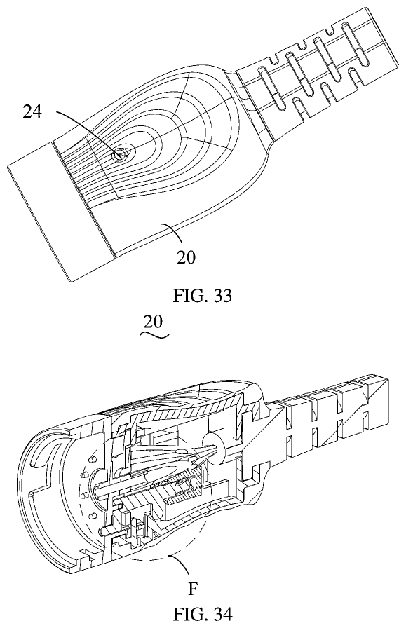

Further, as shown in FIG. 33 to FIG. 34, in one embodiment, the limiting pillar 23a is elastically installed in the handle 20. Through the elastic installation of the limiting pillar 23a, when there is no external force, the limiting pillar 23a extends to such a length that it may enter the limiting hole 400d, thereby limiting the treatment tip 10; when the limiting pillar 23a is subjected to an external force, the limiting pillar 23a moves toward the handle 20, and the limiting pillar 23a exits the limiting hole 400d. Since the limiting pillar 23a is elastically installed on the handle 20, when the treatment tip 10 rotates in cooperation with the handle 20, the end of the treatment tip 10 presses against the limiting pillar 23a. When the limiting pillar 23a is just is aligned with the limiting hole 400d, the limiting pillar 23a rebounds and falls into the limiting hole 400d. At this time, there will be a “pop” sound, indicating that the treatment tip 10 and the handle 20 are installed successfully, otherwise the cooperation between the two fails.

As shown in FIG. 33 to FIG. 34, in one embodiment, the handle 20 is provided with a button 24, and the button 24 is connected to the limiting pillar 23a to drive the movement of the limiting pillar 23a. The user may press button 24 to provide an external force to the limiting pillar 23a, driving the limiting pillar 23a to move inside the handle 20, and the limiting pillar 23a exits the limiting hole 400d, so that the treatment tip 10 may rotate.

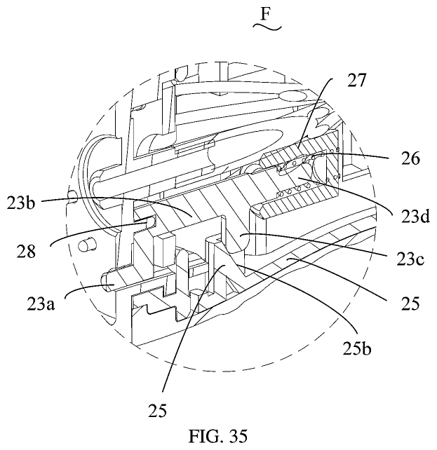

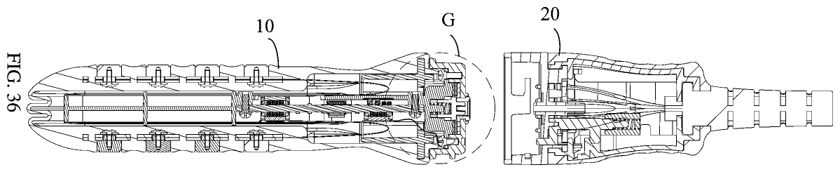

As shown in FIG. 35, in one embodiment, the handle 20 is provided with a boom 25. The surface of the boom 25 is provided with a button 24. A bump 25a is provided on the inside of the boom 25. The bump 25a has a bevel 25b. The bevel 25b extends obliquely from one end close to the installation groove to the direction close to the boom 25. The limiting pillar 23a is provided with a link 23b and a guide block 23c. The link 23b connects the limiting pillar 23a and the guide block 23c. The guide block 23c is located on a side of the link 23b facing the bevel 25b.

As shown in FIG. 32 and FIG. 35, the user presses the button 24, causing the boom 25 to move downward. At this time, the bump 25a has a downward movement force on the guide block 23c. Due to the bevel 25b of the bump 25a, the guide block 23c is pushed to move away from the installation groove 20a, driving the link 23b to move backward, causing the limiting pillar 23a on the link 23b to move towards the inside of the handle 20. In this way, the up and down motion is converted into the axial motion of the handle 20, which cleverly drives the movement of the limiting pillar 23a.

There are many ways to realize the elastic movement of the limiting pillar 23a in the axial direction of the handle 20. As shown in FIG. 35, in one embodiment, the link 23b realizes the elastic movement of the limiting pillar 23a through the elastic piece 26. An installation pillar 27 is installed in the handle 20. The installation pillar 27 is provided with a guide groove for accommodating the elastic piece 26. One end of the elastic piece 26 is in contact with the link 23b, and the other end is in contact with the bottom of the guide groove. In the absence of external force, the elastic piece 26 is in a compressed state, the driving link 23b moves in the direction of the installation groove 20a, and the limiting pillar 23a extends to enter the limiting hole 400d. Under the action of button 24, the driving link 23b moves away from the installation groove 20a, the limiting pillar 23a moves towards the inside of the handle 20, and the elastic piece 26 is compressed and its volume becomes smaller. It may be understood that the elastic piece 26 may be a spring.

In order to facilitate installation and positioning, as shown in FIG. 35, in one embodiment, a guide bar 23d is provided at one end of the link 23b that contacts the elastic piece 26. The outer diameter of the guide bar 23d is smaller than the outer diameter of the link 23b. The elastic piece 26 may be sleeved on the outer periphery of the guide bar 23d. Similarly, as shown in FIG. 35, the back of the bottom of the installation groove 20a is provided with a protrusion pillar 28, and the link 23b is provided with a pillar groove to accommodate the protrusion pillar 28. This not only facilitates the installation and positioning of the link 23b, but also guides the movement of the link 23b to prevent the limiting pillar 23a from deviating from the through hole, so that the limiting pillar 23a may smoothly extend from the through hole when it is reset.

The above are only some embodiments of the present application, and are not intended to limit the scope of the present application. Under the concept of the present application, equivalent structural transformations made according to the description and drawings of the present application, or direct/indirect application in other related technical fields, are included in the scope of the present application.

1. A treatment tip for treating vaginal cavity, wherein the treatment tip is configured to be detachably connected to a handle, and the treatment tip comprises:
a flexible shell being inflatable; and
a support component disposed in the flexible shell to support the flexible shell.

2-3. (canceled)

4. The treatment tip of claim 1, wherein the treatment tip further comprises a hard circuit board and a plurality of electrode sheets; the hard circuit board is disposed on the flexible shell to support the flexible shell, and the electrode sheets are disposed on the hard circuit board and exposed outside an outer peripheral wall of the flexible shell; the plurality of electrode sheets are provided at intervals along a length direction of the flexible shell.

5. The treatment tip of claim 1, wherein the flexible shell is provided with an inflatable cavity, and the support component comprises a support portion extending into the inflatable cavity to support the flexible shell.

6. The treatment tip of claim 5, wherein the treatment tip further comprises an installation seat installed on the flexible shell, and the installation seat is provided with an inflation hole communicated with the inflatable cavity.

7. The treatment tip of claim 6, wherein the treatment tip further comprises a sealing gasket installed on the installation seat, and the sealing gasket comprises a gasket body and a sealing ring; the gasket body is provided on a side of the installation seat away from the handle, and the sealing ring is configured to extend along a wall surface of the inflation hole and is disposed around the inflation hole.

8. The treatment tip of claim 7, wherein the sealing ring is provided with an outer flange.

9. The treatment tip of claim 7, wherein the treatment tip further comprises a sealing member, and the sealing member is elastically abutted against the sealing gasket to seal the inflation hole.

10. The treatment tip of claim 6, wherein the flexible shell is provided with a port communicated with the inflatable cavity, and the port is provided with an extension ring; the installation seat is provided with a receiving groove for receiving the extension ring, and a periphery of the receiving groove is provided with an annular protrusion for abutting against the flexible shell.

11. The treatment tip of claim 10, wherein a plurality of annular protrusions are provided at intervals around a periphery of the installation seat.

12. The treatment tip of claim 10, wherein the treatment tip further comprises an installation member installed at the port; the installation member comprises an extension barrel for connecting the installation seat, and a thimble embedded in the flexible shell.

13. The treatment tip of claim 12, wherein the support component further comprises a base installed on the installation seat; the support portion is provided on the base, and the installation member is configured to pass through the base and is engaged with the installation seat.

14. The treatment tip of claim 13, wherein one of the installation member and the installation seat is provided with a hook piece, and the other one is provided with a buckle piece being engaged with the hook piece.

15. The treatment tip of claim 4, wherein the treatment tip further comprises an adapter plate, and the adapter plate is installed on the support component; the adapter plate is electrically connected to the hard circuit board, and the adapter plate is configured to electrically connect with the handle.

16. A system for treating vaginal cavity, comprising a treatment tip of claim 1 and a handle, wherein the treatment tip is detachably connected to the handle.

17. The system for treating vaginal cavity of claim 16, wherein the handle is provided with a limiting pillar configured to extend into and out of the treatment tip; the treatment tip is provided with a limiting hole for accommodating the limiting pillar, and the limiting hole is offset from a rotation axis of the handle.

18. The system for treating vaginal cavity of claim 16, wherein one of the treatment tip and the handle is provided with a snap block, and the other of the treatment tip and the handle is provided with a snap slot.

19. The system for treating vaginal cavity of claim 16, wherein the handle is provided with an installation groove, and the installation groove is configured to accommodate at least part of the treatment tip; a groove wall of the installation groove is provided with the snap slot, and the treatment tip is provided with the snap block; and
the snap slot comprises a channel slot for the snap block to extend into and a clamping slot extending along a circumference of the handle, and the channel slot is communicated with the clamping slot.

20. The system for treating vaginal cavity of claim 16, wherein the flexible shell is provided with an inflatable cavity, the treatment tip further comprises an installation seat installed on the flexible shell, and the installation seat is provided with an inflation hole communicated with the inflatable cavity; the handle is provided with an abutting pillar extending into the inflation hole, and the abutting pillar is provided with a gas delivery port.

21. The system for treating vaginal cavity of claim 20, wherein the treatment tip further comprises a sealing gasket installed on the installation seat, and the sealing gasket comprises a gasket body and a sealing ring; the gasket body is provided on a side of the installation seat away from the handle, and the sealing ring is configured to extend along a wall surface of the inflation hole and is exposed at the inflation hole.

22. The system for treating vaginal cavity of claim 20, wherein the treatment tip further comprises a sealing member, and the sealing member is elastically abutted against the sealing gasket to seal the inflation hole; in response to that the abutting pillar extends into the inflation hole, the abutting pillar is abutted against the sealing member to open the inflation hole.
